(19) United States
(12) Patent Application Publication
(10) Pub. No.: US 2022/0087632 A1
(43) Pub. Date: Mar. 24, 2022

(54) IMAGING SYSTEM, IMAGING METHOD AND RADIOTHERAPY SYSTEM

(71) Applicant: OUR UNITED CORPORATION, Xi'an City (CN)

(72) Inventors: Hao YAN, Xi'an City (CN); Jinsheng LI, Shenzhen (CN); Fangzheng CHEN, Xi'an City (CN); Daliang LI, Xi'an City (CN)

(21) Appl. No.: 17/483,409

(22) Filed: Sep. 23, 2021

(30) Foreign Application Priority Data
Sep. 24, 2020 (CN) 202011018090.9
Sep. 24, 2020 (CN) 202022122306.8

(51) Int. Cl. A61B 6/00; A61N 5/10

(52) U.S. Cl. CPC A61B 6/5258; A61B 6/4208; A61N 2005/1061; A61B 6/5205; A61N 5/1077; A61B 6/501

(57) ABSTRACT

Embodiments of the present disclosure provide an imaging system, an imaging method, and a radiotherapy system. The imaging system is used for imaging a patient, including: a X-ray tube and a flat-panel detector, the X-ray tube is arranged opposite to the flat-panel detector, the X-ray tube emits X-rays, the flat-panel detector detects the X-rays to generate image data, and a geometric center of the flat-panel detector has an offset relative to an orthographic projection of a focal of the X-ray tube on the flat-panel detector. In the embodiments of the present disclosure, an imaging accuracy obtained based on the image data is better, and an effect of image guidance in tumor treatment is improved.

### CROSS-REFERENCE TO RELATED APPLICATIONS

The present disclosure claims priority to Chinese application No. 202022122306.8 filed on Sep. 24, 2020, and Chinese application No. 202011018090.9 filed on Sep. 24, 2020, which are hereby incorporated by reference in their entireties.

### TECHNICAL FIELD

The present disclosure relates to the field of imaging technology, in particular to an imaging system, an imaging method, and a radiotherapy system.

### BACKGROUND

In the field of imaging technology, a X-ray tube emits X-rays, and a flat-panel detector detects the X-rays emitted by the X-ray tube to generate image data. Based on imaging obtained by the image data, a tumor treatment apparatus treats a tumor in the imaging. However, an imaging system usually has the problem that edge image data obtained based on the image data is blurred, an imaging accuracy is poor, and an effect of tumor treatment is affected.

### SUMMARY

In view of this, one of the technical problems solved by embodiments of the present disclosure is to provide an imaging method, an imaging apparatus, and a radiotherapy system to overcome all or part of the problems existing in the prior art.

An embodiment of the present disclosure provides an imaging system, being used for imaging a patient, including: a X-ray tube and a flat-panel detector, where the X-ray tube is arranged opposite to the flat-panel detector, the X-ray tube emits X-rays, the flat-panel detector detects the X-rays to generate image data, and a geometric center of the flat-panel detector has an offset relative to an orthographic projection of a focal of the X-ray tube on the flat-panel detector.

In a specific embodiment of the present disclosure, an installation position of the X-ray tube and/or the flat-panel detector or a movement apparatus of the flat-panel detector causes the geometric center of the flat-panel detector to have the offset relative to the orthographic projection of the focal of the X-ray tube on the flat-panel detector.

In a specific embodiment of the present disclosure, the movement apparatus is capable of implementing translational movement or rotational movement.

In a specific embodiment of the present disclosure, the movement apparatus sends the offset generated by a movement to an interface for a corresponding orthographic projection algorithm to generate the corresponding image data.

In a specific embodiment of the present disclosure, the algorithm includes an analog orthographic projection algorithm or a reconstruction algorithm.

In a specific embodiment of the present disclosure, the geometric center of the flat-panel detector has the offset in a head-feet direction of the patient relative to the orthographic projection of the focal of the X-ray tube on the flat-panel detector.

In a specific embodiment of the present disclosure, the geometric center of the flat-panel detector has the offset in a direction toward the patient's feet relative to the orthographic projection of the focal of the X-ray tube on the flat-panel detector.

In a specific embodiment of the present disclosure, the geometric center of the flat-panel detector has a first offset in a direction toward the patient's feet relative to the orthographic projection of the focal of the X-ray tube on the flat-panel detector; or,

In a specific embodiment of the present disclosure, the geometric center of the flat-panel detector has the offset in a direction away from an imaging area of interest relative to the orthographic projection of the focal of the X-ray tube on the flat-panel detector.

In a specific embodiment of the present disclosure, if an imaging area of interest is located on the patient's head, the geometric center of the flat-panel detector has a second offset in a direction toward the patient's feet relative to the orthographic projection of the focal of the X-ray tube on the flat-panel detector.

In a specific embodiment of the present disclosure, if an imaging area of interest is located on the patient's body part, the geometric center of the flat-panel detector has a third offset toward an opposite direction to the patient's body part relative to the orthographic projection of the focal of the X-ray tube on the flat-panel detector.

In a specific embodiment of the present disclosure, the imaging area of interest and the geometric center of the flat-panel detector are respectively located on different sides of the orthographic projection of the focal of the X-ray tube on the flat-panel detector.

In a specific embodiment of the present disclosure, the first offset, the second offset, or the third offset is less than or equal to ¾ of a length or a width of the flat-panel detector.

In a specific embodiment of the present disclosure, the first offset, the second offset, or the third offset is less than or equal to ½ of a length or a width of the flat-panel detector.

In a specific embodiment of the present disclosure, the first offset, the second offset, or the third offset is less than or equal to 200 mm.

In a specific embodiment of the present disclosure, the first offset, the second offset, or the third offset is less than or equal to 100 mm.

The present disclosure also provides a radiotherapy system, including a tumor treatment apparatus and the imaging system as described in any one of the above embodiments.

In a specific embodiment of the present disclosure, an imaging area of the imaging system and a treatment area of the tumor treatment apparatus at least partially overlap.

The present disclosure also provides an imaging method, applied to a X-ray tube and a flat-panel detector arranged oppositely, the method including:

the X-ray tube emitting X-rays, and an orthographic projection of a focus of the X-ray tube on the flat-panel detector having an offset relative to a geometric center of the flat-panel detector; and

the flat-panel detector detecting the X-rays to generate image data.

In a specific embodiment of the present disclosure, the flat-panel detector detecting the X-rays to generate image data, includes:

acquiring the offset; and

generating the corresponding image data, based on the offset and a corresponding orthographic projection algorithm.

In a specific embodiment of the present disclosure, the generating the corresponding image data, based on the offset and a corresponding orthographic projection algorithm includes: generating a corresponding imaging mode based on the offset; and generating the corresponding image data, based on the imaging mode and an algorithm corresponding to the imaging mode.

In a specific embodiment of the present disclosure, the geometric center of the flat-panel detector has the offset in a head-feet direction of the patient relative to the orthographic projection of the focal of the X-ray tube on the flat-panel detector.

In a specific embodiment of the present disclosure, the geometric center of the flat-panel detector has an offset in a direction toward the patient's feet relative to the orthographic projection of the focal of the X-ray tube on the flat-panel detector.

In a specific embodiment of the present disclosure, the geometric center of the flat-panel detector has a first offset relative to the orthographic projection of the focal of the X-ray tube on the flat-panel detector.

In a specific embodiment of the present disclosure, the geometric center of the flat-panel detector has the offset in a direction away from an imaging area of interest relative to the orthographic projection of the focal of the X-ray tube on the flat-panel detector.

In a specific embodiment of the present disclosure, if an imaging area of interest is located on the patient's head, the geometric center of the flat-panel detector has a second offset a direction toward the patient's feet relative to the orthographic projection of the focal of the X-ray tube on the flat-panel detector.

In a specific embodiment of the present disclosure, if an imaging area of interest is located on the patient's body part, the geometric center of the flat-panel detector has a third offset toward an opposite direction to the patient's body part relative to the orthographic projection of the focal of the X-ray tube on the flat-panel detector.

In a specific embodiment of the present disclosure, the imaging area of interest and the geometric center of the flat-panel detector are respectively located on different sides of the orthographic projection of the focal of the X-ray tube on the flat-panel detector.

In a specific embodiment of the present disclosure, the first offset, the second offset, or the third offset is less than or equal to ¾ of a length or a width of the flat-panel detector.

In a specific embodiment of the present disclosure, the first offset, the second offset, or the third offset is less than or equal to ½ of a length or a width of the flat-panel detector.

It can be seen from the above technical solution that the imaging system provided by the embodiments of the present disclosure includes the X-ray tube that emits X-rays and the flat-panel detector that detects the X-rays to generate image data. In the embodiments of the present disclosure, the geometric center of the flat-panel detector has the offset relative to the orthographic projection of the focal of the X-ray tube on the flat-panel detector, so as to avoid that the orthographic projection of the X-rays on the flat-panel detector coincides with the geometric center of the flat-panel detector, resulting in poor quality of some edge image data obtained by the flat-panel detector based on the image data. In the embodiments of the present disclosure, an imaging accuracy obtained based on the image data is better, and an effect of image guidance in tumor treatment is improved.

### BRIEF DESCRIPTION OF THE DRAWINGS

In order to more clearly illustrate the technical solution in embodiments of the present disclosure or the prior art, the following will briefly introduce the accompanying drawings that need to be used in the description of the embodiments or the prior art. Obviously, the accompanying drawings in the following description are only some of the embodiments described in the embodiments of the present disclosure. For those of ordinary skill in the art, other accompanying drawings may also be obtained based on these accompanying drawings.

FIG. 1 is a schematic diagram of a conventional imaging system;

FIG. 2 is a schematic diagram of an imaging system according to an embodiment of the present disclosure;

FIG. 3 is a schematic diagram of the imaging system according to another embodiment of the present disclosure;

FIG. 4 is a schematic diagram of the imaging system according to yet another embodiment of the present disclosure;

FIG. 5 is a flowchart of an imaging method according to an embodiment of the present disclosure;

FIG. 6 is a flowchart of step 502 of the imaging method according to another embodiment of the present disclosure; and

FIG. 7 is a flowchart of step 5022 of the imaging method according to yet another embodiment of the present disclosure.

### DETAILED DESCRIPTION OF EMBODIMENTS

In order to enable those skilled in the art to better understand the technical solution in embodiments of the present disclosure, the technical solution in the embodiments of the present disclosure will be clearly and completely described below in conjunction with the accompanying drawings in the embodiments of the present disclosure. Obviously, the described embodiments are only a part of the embodiments of the present disclosure, rather than all the embodiments. Based on the embodiments in the embodiments of the present disclosure, all other embodiments obtained by those of ordinary skill in the art should fall within the protection scope of the embodiments of the present disclosure.

Referring to FIG. 1, generally, the imaging system includes: a X-ray tube 11 and a flat-panel detector 12, the X-ray tube 11 is arranged opposite to the flat-panel detector 12, the X-ray tube 11 emits X-rays, and the flat-panel detector 12 detects the X-rays to generate image data. An orthographic projection range of the X-rays emitted by the X-ray tube 11 on the flat-panel detector 12 is defined by a ray radius range B1 to a patient's head and a ray radius range B2 to the patient's feet, and the ray radius range B1 to the patient's head is equal to the ray radius range B2 to the patient's feet. The orthographic projection of the focal of the X-ray tube 11 on the flat-panel detector 12 coincides with the geometric center of the flat-panel detector 12, and some edge image data obtained by detection by the flat-panel detector 12 is blurred, resulting in poor imaging accuracy obtained based on the image data, which affects an effect of tumor treatment.

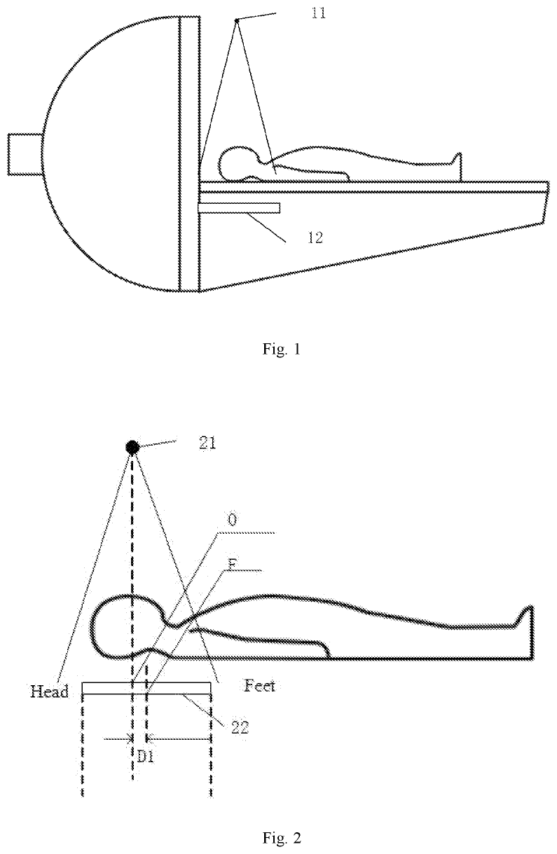

Referring to FIG. 2, an embodiment of the present disclosure provides an imaging system, for imaging a patient. The imaging system includes: a X-ray tube 21 and a flat-panel detector 22. The X-ray tube 21 is arranged opposite to the flat-panel detector 22, the X-ray tube 21 emits X-rays, the flat-panel detector 22 detects the X-rays to generate image data, and a geometric center F of the flat-panel detector 22 has an offset relative to an orthographic projection 0 of a focal of the X-ray tube 21 on the flat-panel detector 22.

The imaging system provided by the embodiments of the present disclosure includes the X-ray tube that emits X-rays and the flat-panel detector that detects the X-rays to generate image data. In the embodiments of the present disclosure, the geometric center of the flat-panel detector has the offset relative to the orthographic projection of the focal of the X-ray tube on the flat-panel detector, so as to avoid that the orthographic projection of the X-rays on the flat-panel detector coincides with the geometric center of the flat-panel detector, resulting in poor quality of some edge image data obtained by the flat-panel detector based on the image data. In the embodiments of the present disclosure, an imaging accuracy obtained based on the image data is better, and an effect of image guidance in tumor treatment is improved.

In a specific implementation of the embodiments of the present disclosure, an installation position of the X-ray tube 21 and/or the flat-panel detector 22 or a movement apparatus of the flat-panel detector 22 (not shown in the figure) causes the geometric center of the flat-panel detector 22 to have the offset relative to the orthographic projection of the focal of the X-ray tube 21 on the flat-panel detector 22.

In the embodiments of the present disclosure, the installation position of the X-ray tube 21 and/or the flat-panel detector 22 may realize that the geometric center of the flat-panel detector 22 has the offset relative to the orthographic projection of the focal of the X-ray tube 21 on the flat-panel detector 22. In the embodiments of the present disclosure, the movement apparatus of the flat-panel detector 22 (not shown in the figure) may also realize that the geometric center of the flat-panel detector 22 has the offset relative to the orthographic projection of the focal of the X-ray tube 21 on the flat-panel detector 22. The embodiments of the present disclosure may be applied to various imaging systems with X-ray tubes and flat-panel detectors, and only the installation position or the movement apparatus of the flat-panel detector 22 is required to realize that the geometric center of the flat-panel detector has the offset relative to the orthographic projection of the focal of the X-rays emitted by the X-ray tube on the flat-panel detector. The embodiments of the present disclosure have a wide range of applications and are easy to implement.

Specifically, the movement apparatus may implement translational movement or rotational movement. Therefore, the embodiments of the present disclosure may be applied to an imaging system of the flat-panel detector in translational movement, and also to an imaging system of the flat-panel detector in rotational movement.

In another specific implementation of the embodiments of the present disclosure, the movement apparatus sends the offset generated by a movement to an interface for a corresponding orthographic projection algorithm to generate the corresponding image data.

Therefore, the embodiments of the present disclosure may adopt different orthographic projection algorithms based on different offsets generated by the movement of the movement apparatus, so as to obtain more accurate image data and realize better image guidance.

Specifically, the algorithm includes an analog orthographic projection algorithm or a reconstruction algorithm.

Therefore, the embodiments of the present disclosure adopt different analog orthographic projection algorithms or reconstruction algorithms to obtain more accurate image data, so as to realize better image guidance.

In another specific implementation of the embodiments of the present disclosure, referring to FIG. 2, the geometric center of the flat-panel detector has the offset in a head-feet direction of the patient relative to the orthographic projection of the focal of the X-ray tube on the flat-panel detector.

The geometric center of the flat-panel detector has the offset in a direction toward the patient's feet relative to the orthographic projection of the focal of the X-ray tube on the flat-panel detector.

Specifically, the geometric center of the flat-panel detector has a first offset in a direction toward the patient's feet relative to the orthographic projection of the focal of the X-ray tube on the flat-panel detector.

Further, as an example, the geometric center of the flat-panel detector has an offset in a left-right direction of the patient relative to the orthographic projection of the focal of the X-ray tube on the flat-panel detector.

In another specific implementation of the embodiments of the present disclosure, the geometric center of the flat-panel detector has the offset in a direction away from an imaging area of interest relative to the orthographic projection of the focal of the X-ray tube on the flat-panel detector.

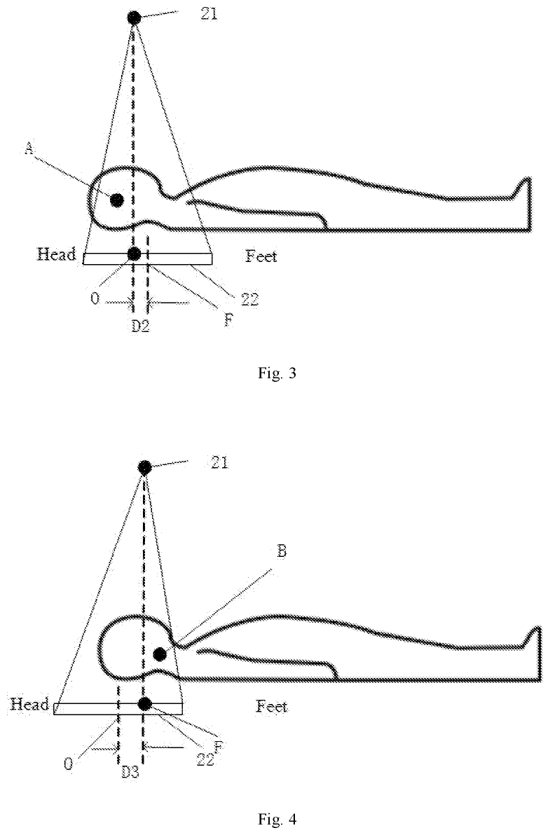

For example, referring to FIG. 3, if the imaging area of interest is located on the patient's head A, the geometric center of the flat-panel detector has a second offset D2 in a direction toward the patient's feet relative to the orthographic projection of the focal of the X-ray tube on the flat-panel detector.

Therefore, the embodiments of the present disclosure reduce blurring of edge image data of the imaging area of interest, that is, the edge image data of the patient's head, ensure that the imaging area of interest obtains more accurate edge image data, and improve the effect of image guidance.

For example, referring to FIG. 4, if the imaging area of interest is located on the patient's body part B, the geometric center of the flat-panel detector has a third offset D3 toward an opposite direction to the patient's body part relative to the orthographic projection of the focal of the X-ray tube on the flat-panel detector.

Therefore, the embodiments of the present disclosure reduce blurring of edge image data of the imaging area of interest, that is, the edge image data of the patient's body part, ensure that the imaging area of interest obtains more accurate edge image data, and improve the effect of image guidance.

The imaging area of interest and the geometric center of the flat-panel detector are respectively located on different sides of the orthographic projection of the focal of the X-ray tube on the flat-panel detector.

In another specific implementation of the embodiments of the present disclosure, the first offset, the second offset, or the third offset is less than or equal to ¾ of a length or a width of the flat-panel detector.

The first offset, the second offset, or the third offset in the embodiments of the present disclosure are set so that the flat-panel detector can obtain better image data.

In another specific implementation of the embodiments of the present disclosure, the first offset, the second offset, or the third offset is less than or equal to ½ of a length or a width of the flat-panel detector.

The first offset, the second offset, or the third offset in the embodiments of the present disclosure are set so that the flat-panel detector can obtain better image data.

In another specific implementation of the embodiments of the present disclosure, the first offset, the second offset, or the third offset is less than or equal to 200 mm.

The first offset, the second offset, or the third offset in the embodiments of the present disclosure are set so that the flat-panel detector can obtain better image data.

In another specific implementation of the embodiments of the present disclosure, the first offset, the second offset, or the third offset is less than or equal to 100 mm.

The first offset, the second offset, or the third offset in the embodiments of the present disclosure are set so that the flat-panel detector can obtain better image data.

An embodiment of the present disclosure also provides a radiotherapy system, including a tumor treatment apparatus and the imaging system as described in any one of the above embodiments.

In a specific implementation of the embodiments of the present disclosure, an imaging area of the imaging system and a treatment area of the tumor treatment apparatus at least partially overlap.

The imaging system provided by the embodiments of the present disclosure includes the X-ray tube that emits X-rays and the flat-panel detector that detects the X-rays to generate image data. In the embodiments of the present disclosure, the geometric center of the flat-panel detector has the offset relative to the orthographic projection of the focal of the X-ray tube on the flat-panel detector, so as to avoid that the orthographic projection of the X-rays on the flat-panel detector coincides with the geometric center of the flat-panel detector, resulting in poor quality of some edge image data obtained by the flat-panel detector based on the image data. In the embodiments of the present disclosure, an imaging accuracy obtained based on the image data is better, and an effect of image guidance in tumor treatment is improved.

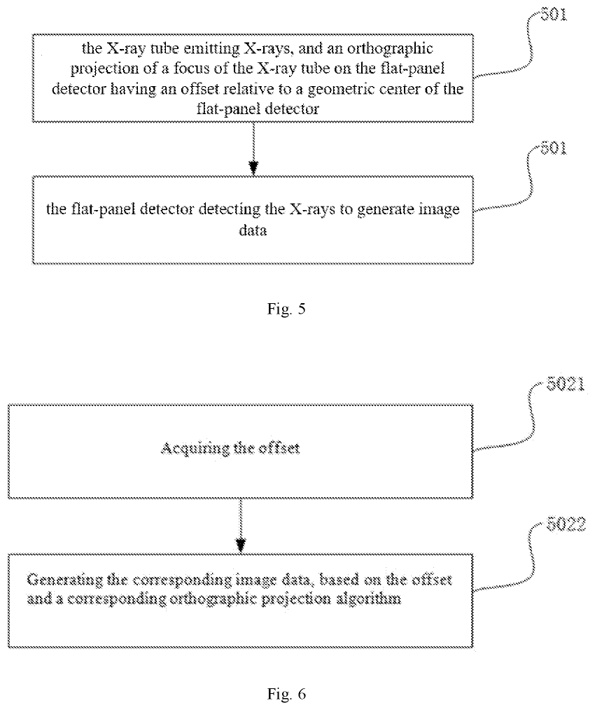

The present disclosure also provides an imaging method, applied to a X-ray tube and a flat-panel detector arranged oppositely. Referring to FIG. 5, the method includes:

Step 501, the X-ray tube emitting X-rays, and an orthographic projection of a focus of the X-ray tube on the flat-panel detector having an offset relative to a geometric center of the flat-panel detector.

Step 502, the flat-panel detector detecting the X-rays to generate image data.

The imaging system provided by the embodiments of the present disclosure includes the X-ray tube that emits X-rays and the flat-panel detector that detects the X-rays to generate image data. In the embodiments of the present disclosure, the geometric center of the flat-panel detector has the offset relative to the orthographic projection of the focal of the X-ray tube on the flat-panel detector, so as to avoid that the orthographic projection of the X-rays on the flat-panel detector coincides with the geometric center of the flat-panel detector, resulting in poor quality of some edge image data obtained by the flat-panel detector based on the image data. In the embodiments of the present disclosure, an imaging accuracy obtained based on the image data is better, and an effect of image guidance in tumor treatment is improved.

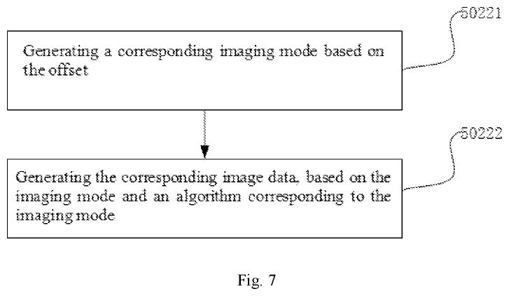

In a specific implementation of the embodiments of the present disclosure, referring to FIG. 6, the step 502, includes:

Step 5021, acquiring the offset.

Step 5022, generating the corresponding image data, based on the offset and a corresponding orthographic projection algorithm.

Therefore, the embodiments of the present disclosure may adopt different orthographic projection algorithms based on different offsets generated by the movement of the movement apparatus, so as to obtain more accurate image data to realize better image guidance.

Specifically, the algorithm includes an analog orthographic projection algorithm or a reconstruction algorithm.

Therefore, the embodiments of the present disclosure adopt different analog orthographic projection algorithms or reconstruction algorithms to obtain more accurate image data, so as to realize better image guidance.

In another specific implementation of the embodiments of the present disclosure, referring to FIG. 7, the step 5022, includes:

Step 50221, generating a corresponding imaging mode based on the offset.

Step 50222, generating the corresponding image data, based on the imaging mode and an algorithm corresponding to the imaging mode.

In a specific implementation of the embodiments of the present disclosure, the geometric center of the flat-panel detector has the offset in a head-feet direction of the patient relative to the orthographic projection of the focal of the X-ray tube on the flat-panel detector.

In a specific implementation of the embodiments of the present disclosure, the geometric center of the flat-panel detector is offset in a direction toward the patient's feet relative to the orthographic projection of the focal of the X-ray tube on the flat-panel detector.

In a specific implementation of the embodiments of the present disclosure, referring to FIG. 2, the geometric center of the flat-panel detector has a first offset D1 relative to the orthographic projection of the focal of the X-ray tube on the flat-panel detector.

In a specific implementation of the embodiments of the present disclosure, the geometric center of the flat-panel detector has the offset in a direction away from an imaging area of interest relative to the orthographic projection of the focal of the X-ray tube on the flat-panel detector.

In a specific implementation of the embodiments of the present disclosure, referring to FIG. 3, if the imaging area of interest is located on the patient's head, the geometric center of the flat-panel detector has a second offset D2 in a direction toward the patient's feet relative to the orthographic projection of the focal of the X-ray tube on the flat-panel detector.

Therefore, the embodiments of the present disclosure reduce blurring of edge image data of the imaging area of interest, that is, the edge image data of the patient's head, ensure that the imaging area of interest obtains more accurate edge image data, and improve the effect of image guidance.

In a specific implementation of the embodiments of the present disclosure, referring to

FIG. 4, if the imaging area of interest is located on the patient's body part, the geometric center of the flat-panel detector has a third offset D3 toward an opposite direction to the patient's body part relative to the orthographic projection of the focal of the X-ray tube on the flat-panel detector.

Therefore, the embodiments of the present disclosure reduce blurring of edge image data of the imaging area of interest, that is, the edge image data of the patient's body part, ensure that the imaging area of interest obtains more accurate edge image data, and improve the effect of image guidance.

In a specific implementation of the embodiments of the present disclosure, the imaging area of interest and the geometric center of the flat-panel detector are respectively located on different sides of the orthographic projection of the focal of the X-ray tube on the flat-panel detector.

In a specific implementation of the embodiments of the present disclosure, the first offset, the second offset, or the third offset is less than or equal to ¾ of a length or a width of the flat-panel detector.

The first offset, the second offset, or the third offset in the embodiments of the present disclosure are set so that the flat-panel detector can obtain better image data.

In a specific implementation of the embodiments of the present disclosure, the first offset, the second offset, or the third offset is less than or equal to ½ of a length or a width of the flat-panel detector.

The first offset, the second offset, or the third offset in the embodiments of the present disclosure are set so that the flat-panel detector can obtain better image data.

The apparatus embodiments described above are merely illustrative, where the units described as separate components may or may not be physically separated, and the components displayed as units may or may not be physical units, that is, they may be located in one place, or may be distributed to a plurality of network units. Some or all of the modules may be selected according to actual needs to achieve the purpose of the solution of the embodiment. Those of ordinary skill in the art may understand and implement it without creative work.

Finally, it should be noted that the above embodiments are only used to illustrate the technical solution of the embodiments of the present disclosure, but rather than restrict them; although the present disclosure has been described in detail with reference to the foregoing embodiments, those of ordinary skill in the art should understand that: the technical solution recorded in the foregoing embodiments can still be modified, or some of the technical features can be equivalently replaced; however, these modifications or replacements do not cause the essence of the corresponding technical solution to deviate from the spirit and scope of the technical solution of the embodiments of the present disclosure.

What is claimed is:

1. An imaging system for imaging a patient, comprising: a X-ray tube and a flat-panel detector, the X-ray tube being arranged opposite to the flat-panel detector, wherein the X-ray tube emits X-rays, the flat-panel detector detects the X-rays to generate image data, and a geometric center of the flat-panel detector has an offset relative to an orthographic projection of a focal of the X-ray tube on the flat-panel detector.

2. The imaging system according to claim 1, wherein:
an installation position of the X-ray tube and/or the flat-panel detector causes the geometric center of the flat-panel detector to have the offset relative to the orthographic projection of the focal of the X-ray tube on the flat-panel detector; or
the X-ray tube or the flat-panel detector comprises a movement apparatus, and the movement apparatus of the X-ray tube or the flat-panel detector causes the geometric center of the flat-panel detector to have the offset relative to the orthographic projection of the focal of the X-ray tube on the flat-panel detector.

3. The imaging system according to claim 2, wherein the movement apparatus is capable of implementing translational movement or rotational movement.

4. The imaging system according to claim 1, wherein the offset generated by a movement is sent to an interface for a corresponding orthographic projection algorithm to generate a corresponding image data.

5. The imaging system according to claim 1, wherein the geometric center of the flat-panel detector has the offset in a head-feet direction of the patient relative to the orthographic projection of the focal of the X-ray tube on the flat-panel detector.

6. The imaging system according to claim 5, wherein the geometric center of the flat-panel detector has the offset in a direction toward the patient's feet relative to the orthographic projection of the focal of the X-ray tube on the flat-panel detector.

7. The imaging system according to claim 5, wherein:
the geometric center of the flat-panel detector has a first offset in a direction toward the patient's feet relative to the orthographic projection of the focal of the X-ray tube on the flat-panel detector; or,
if an imaging area of interest is located on the patient's head, the geometric center of the flat-panel detector has a second offset in the direction toward the patient's feet relative to the orthographic projection of the focal of the X-ray tube on the flat-panel detector; or,
if an imaging area of interest is located on the patient's body part, the geometric center of the flat-panel detector has a third offset toward an opposite direction to the patient's body part relative to the orthographic projection of the focal of the X-ray tube on the flat-panel detector.

8. The imaging system according to claim 5, wherein the geometric center of the flat-panel detector has the offset in a direction away from an imaging area of interest relative to the orthographic projection of the focal of the X-ray tube on the flat-panel detector.

9. The imaging system according to claim 8, wherein the imaging area of interest and the geometric center of the flat-panel detector are respectively located on different sides of the orthographic projection of the focal of the X-ray tube on the flat-panel detector.

10. The imaging system according to claim 7, wherein:
the first offset, the second offset, or the third offset is less than or equal to ¾ of a length or a width of the flat-panel detector;
the first offset, the second offset, or the third offset is less than or equal to ½ of a length or a width of the flat-panel detector;
the first offset, the second offset, or the third offset is less than or equal to 200 mm; or the first offset, the second offset, or the third offset is less than or equal to 100 mm.

11. A radiotherapy system, comprising a tumor treatment apparatus and an imaging system;
the imaging system being used for imaging a patient, and comprising: a X-ray tube and a flat-panel detector, the X-ray tube being arranged opposite to the flat-panel detector, wherein the X-ray tube emits X-rays, the flat-panel detector detects the X-rays to generate image data, and a geometric center of the flat-panel detector has an offset relative to an orthographic projection of a focal of the X-ray tube on the flat-panel detector.

12. The radiotherapy system according to claim 11, wherein an imaging area of the imaging system and a treatment area of the tumor treatment apparatus at least partially overlap.

13. An imaging method, applied to a X-ray tube and a flat-panel detector arranged oppositely, the method comprising:
the X-ray tube emitting X-rays, and an orthographic projection of a focus of the X-ray tube on the flat-panel detector having an offset relative to a geometric center of the flat-panel detector; and
the flat-panel detector detecting the X-rays to generate image data.

14. The imaging method according to claim 13, wherein the flat-panel detector detecting the X-rays to generate image data, comprises:
acquiring the offset; and
generating a corresponding image data, based on the offset and a corresponding orthographic projection algorithm.

15. The imaging method according to claim 14, wherein, generating a corresponding imaging mode based on the offset; and
generating the corresponding image data, based on the imaging mode and an algorithm corresponding to the imaging mode.

16. The imaging method according to claim 13, wherein the geometric center of the flat-panel detector has the offset in a head-feet direction of the patient relative to the orthographic projection of the focal of the X-ray tube on the flat-panel detector.

17. The imaging method according to claim 16, wherein the geometric center of the flat-panel detector is offset in a direction toward the patient's feet relative to the orthographic projection of the focal of the X-ray tube on the flat-panel detector.

18. The imaging method according to claim 13, wherein:
the geometric center of the flat-panel detector has a first offset relative to the orthographic projection of the focal of the X-ray tube on the flat-panel detector; or,
if an imaging area of interest is located on the patient's head, the geometric center of the flat-panel detector has a second offset in a direction toward the patient's feet relative to the orthographic projection of the focal of the X-ray tube on the flat-panel detector; or,
if an imaging area of interest is located on the patient's body part, the geometric center of the flat-panel detector has a third offset toward an opposite direction to the patient's body part relative to the orthographic projection of the focal of the X-ray tube on the flat-panel detector.

19. The imaging method according to claim 13, wherein the geometric center of the flat-panel detector has the offset in a direction away from an imaging area of interest relative to the orthographic projection of the focal of the X-ray tube on the flat-panel detector.

20. The imaging method according to claim 13, wherein the imaging area of interest and the geometric center of the flat-panel detector are respectively located on different sides of the orthographic projection of the focal of the X-ray tube on the flat-panel detector.

21. The imaging method according to claim 18, wherein the first offset, the second offset, or the third offset is less than or equal to ¾ of a length or a width of the flat-panel detector; or
the first offset, the second offset, or the third offset is less than or equal to ½ of a length or a width of the flat-panel detector.
